(19) United States
(12) Patent Application Publication
(10) Pub. No.: US 2026/0314960 A1
(43) Pub. Date: Oct. 15, 2026

(54) CURRENT MEASUREMENT DEVICE FOR AN ANALYTE MONITORING SYSTEM

(71) Applicant: GLUCORX TECHNOLOGIES LIMITED, Dingwall Highland (GB)

(72) Inventors: James Iain Rodgers, Dingwall Highland (GB); David Geoffrey Pitts, Dingwall Highland (GB); Paul Vincent Knight, Dingwall Highland (GB); Ivan Buliev, Dingwall Highland (GB); Nikolay Nikolov, Dingwall Highland (GB)

(21) Appl. No.: 19/485,015

(22) PCT Filed: May 15, 2024
(86) PCT No.: PCT/EP2024/063425

§ 371 Date: Nov. 17, 2025

(30) Foreign Application Priority Data
May 15, 2023 (EP) 2307226.7

(51) Int. Cl. A61B 5/145; A61B 5/1486; G01R 19/00

(52) U.S. Cl. CPC A61B 5/14532; A61B 5/14865; G01R 19/0023

(57) ABSTRACT

A current measurement device for an analyte monitoring system, the measurement device comprising: a skin-implantable amperometric sensor connected to a Resettable Integrator Circuit (RIC) comprising: a capacitor configured to be charged by an electrical current flowing from the amperometric sensor, a voltage measurement system configured to sample a voltage at the RIC output, the sampled voltage being indicative of the charge in the capacitor and indicative of a current flowing from the amperometric sensor into RIC; and a controller configured to: (i) start a measurement cycle by charging the capacitor, and (ii) end the measurement cycle by resetting the RIC to discharge the capacitor.

### TECHNICAL FIELD

The invention relates to a current measurement device for an analyte monitoring system.

### BACKGROUND

Traditionally, monitoring blood glucose levels of a patient involved a process where a finger prick blood test obtained a small drop of blood that was placed on a test strip that inserted into glucometer. The glucometer read the strip and provided a digital reading of the individual's blood sugar level.

Recently, finger prick blood tests have been replaced by insertable (implantable), in vivo, analyte sensors that are inserted into the skin of the patient where they remain at all times, enabling substantially continuous measurements to be taken, which is advantageous compared to finger prick tests that provide only snapshot readings at a small number of times a day. These types of implantable analyte sensors are typically coupled to a sensor module having a housing placed on the surface of the patient's skin. The sensor module control electronics process measurement signals from the inserted sensor and transmit any relevant information to, for example, the patient's smartphone or other mobile device. These types of systems are sometimes known as continuous analyte monitoring systems.

Typically, continuous analyte monitoring systems require the analyte sensor to be replaced at predetermined intervals and this may require the patient to insert the analyte sensor themselves in an unsupervised environment. In order to simplify this process, known analyte monitoring systems are provided with an inserter device which applies a predetermined amount of force to the insertable analyte sensor to safely insert it into the patient's skin, and at the same time to position any accompanying sensor module at the surface of the patient's skin. EP2393417B1 proposes a continuous analyte monitoring system.

In known continuous analyte monitoring systems, such as that of EP2393417B1, the sensor module is initially inside of the inserter device. An adhesive pad or patch (sometimes referred to as an epidermal support patch) is provided on the skin-facing end of the inserter device. During use, the inserter device inserts the analyte sensor into the skin, places the sensor module onto the adhesive pad which is thereby secured to the patient's skin.

The circuitry in the sensor module of known continuous analyte monitoring systems is powered by a battery, which can only power the sensor module for a limited period of time before it needs to be replaced.

Skin-implantable sensors (that is, the part that is implanted into the skin and generates an output current signal based on the concentration of the analyte being measured) may have a variety of sizes, each with advantages and disadvantages. The smaller a sensor is, the more comfortable it is for the patient to insert in their skin but the smaller and noisier the current output that is produced so the less accurate the analyte concentration measurement. Conversely, a larger sensor produces a larger output current signal to focus on accuracy because the signal to noise ratio is higher but this is less comfortable for the user, especially over prolonged periods of time.

There is a need for an improved current measurement device.

### SUMMARY

In general terms, the present disclosure solves the problem that it is difficult to get accurate analyte concentration measurements from small current output signals of skin-implantable sensors that focus on comfort instead of accuracy, e.g. because they are smaller than sensors that focus on accuracy. Specifically, this problem is solved in a power efficient way which ensures that battery life of the battery powering any circuitry is increased. This reduces the frequency at which a patient has remove and re-implant a sensor when a battery needs to be replaced.

Generally, a low power and low current analogue front end including an amperometric sensor is provided. The front end and/or sensor may comprise a very low power Resettable Integrator Circuit (hereafter referred to as RIC). The RIC is first reset to start a cycle in which sensor current is accumulated as a capacitor is charged and the resulting output voltage periodically sampled at defined intervals over the charging period, before being reset and the process repeated. During the measurement cycle the RIC converts the average sensor current over the sampling period into a proportional output voltage. This is periodically sampled, together with information about the sampling period, to determine a charging rate that is indicative of the average current flowing from the amperometric sensor, and thus indicative of the concentration of the analyte being measured. The higher the current from the amperometric sensor, the quicker the capacitor will charge and the higher the voltage will be when sampled and vice versa. Once the voltage has been sampled to make the final current estimate, the RIC is reset to end the measurement cycle and the capacitor discharged. At the next predetermined measurement cycle start time, the above cycle is repeated. The timing of the voltage sampling and current estimates is determined by a controller.

By only sampling the RIC output at intermittent times, the measurement circuitry of the controller is mostly idle and the controller can accordingly sleep for the vast majority of the time, thus using substantially less power than would otherwise be the case. For example, using one example embodiment of the device of the present disclosure to repeat measurement cycles only every 60 seconds for a 20 day period results in only around 2100 seconds of microprocessor awake time and accordingly allows the device to continuously operate for this time without requiring replacement of the (in this non-limiting example) CR1220 lithium button cell battery. In contrast, without intermittent sampling of the RIC output, the same controller would be awake continuously and the device would require a replacement battery within around 7 hours.

The timing at which the final voltage sampling is made to obtain the final current estimate for a given measurement cycle is set dynamically and algorithmically. Specifically, the timing of the voltage measurement sampling at the capacitor to obtain the analyte concentration estimation is set to occur at a time before the capacitor reaches saturation but long enough after a measurement cycle starts to ensure there is sufficient charge in the capacitor for the signal to noise ratio to be strong. Thus, regardless of how small or large an output current signal of the amperometric sensor is over different time periods, the device will be able to dynamically adjust when it makes the final voltage sampling and thus current estimate within the measurement cycle to always be before RIC saturation is reached, but long enough after the start of the measurement cycle to ensure there is enough charge in the capacitor for a high signal to noise ratio.

Accordingly, moving the time when the final voltage sampling is performed towards the end of the measurement cycle allows the charge held in the capacitor to be higher when the voltage is sampled and this offers higher signal to noise ratios at lower currents from the amperometric sensor than would otherwise be obtained. Specifically, this allows very small currents to be reliably measured with low cost components, and high frequency noise to be automatically filtered out at the analogue front-end because the RIC effectively operates as a low-pass filter, rather than computational filtering using a combination of high frequency sampling and numerical averaging. This again, minimises power utilisation as the power consumption of low cost analogue components is typically much lower than a microprocessor.

Conversely, in the event that higher currents flow from the amperometric sensor, moving the time when the final voltage sampling is performed towards the beginning of the measurement cycle allows the voltage to be sampled in good time before the RIC reaches saturation, which occurs more quickly when the current is higher. The device accordingly allows for a wider dynamic range of different currents without saturating, which could result in inaccurate readings and may also cause instability in the fixed input potential being applied to the sensor in the analogue front end circuit.

It is envisaged that the current measurement device of the present disclosure can be used for example for control and measurement of a transcutaneous glucose sensor configured to provide a current reading at, for example, measurement cycles of one minute intervals. In such a situation for the use case of glucose measurements, it has been found there is little advantage to be gained by measuring more frequently than one minute as the changes in glucose concentration and time required to react to these are such that more frequent measurements would have almost no additional utility.

Considering now how the voltage sampling timing of the RIC may be dynamically and algorithmically set, generally the timing will be set based on how quickly the capacitor (of the RIC) is charging and therefore how long it will take for the RIC to saturate. As described above, the longer the device waits to make the final voltage sampling, the more full the capacitor will be when the voltage is sampled and thus the more accurate the current estimate will be for a very low input current signal. Conversely, if the RIC is saturated, or if the capacitor has only just started charging again after being discharged, the measurement will be inaccurate. Thus, the present disclosure determines an optimum voltage sampling time during a measurement cycle to ensure that voltages are not sampled when the RIC is saturated or when the capacitor has only just started charging.

Determining the optimum voltage sample timing may be based on empirically and/or theoretically determined knowledge of the characteristics of the circuitry and measurement components. For example, if determined theoretically, the capacitance, resistance and voltage measurement capability of the low cost components employed may be numerically modelled and the theoretical charge time of the RIC's capacitor estimated for different charge rates. If determined empirically, the system's performance information may be monitored over a number of measurement cycles.

The algorithm to determine the optimum final sample voltage timing accepts as its input sampled voltages from very early on in a measurement cycle, and/or final sampled voltages from previous measurement cycles, and/or the current estimates, capacitor charge rate estimates, or other parameters derived therefrom, for example the rate of change of voltage over time, dV/dt, for the sample time, all of which may accordingly take into account the known capacitance of the capacitor and other components of the circuitry. In some implementations dV/dt may be estimated, for example by computing a change in voltage over a given period of time (ΔV/Δt). The time period (Δt) interval may be selected such that the computed ΔV/At provides an estimate of the rate of change with an acceptable degree of accuracy given the rate of change.

It will be understood that an acceptable accuracy for the estimated rate of change may vary depending on the specific implementation and usage of the device. For example, the device may be configured to provide an estimated rate of change ΔV/At that is within 0.1% to 1% of the true rate of change of voltage over time, dV/dt, such as 0.1%, 0.2%, 03%, 0.4%, 0.5%, 0.6%, 0.7%, 0.8%, 0.9% or 1%. Alternatively, implementations may require an accuracy higher than 0.1% (e.g. 0.01%) or allow for an accuracy lower than 1% (e.g. 2%). In one example implementation, the device may be configured to provide current measurements with an accuracy of 0.4% or less. In another example implementation, the current measurements may be accurate to within ±1 to 10 pA, for example ±1 pA, ±2 pA, ±3 pA, ±4 pA, ±5 pA, ±6 pA, ±7 pA, ±8 pA, ±9 pA or ±10 pA. Alternatively, implementations may require an accuracy higher than ±1 pA (e.g. ±0.1 pA) or allow for an accuracy lower than ±10 pA (e.g. ±100 pA). It will be understood that the acceptable accuracy of the measurements may be defined in both absolute and relative terms. For example, in a further implementation the device may be configured to operate at a current range of 0 to 300 nA, and the device may be further configured to provide current measurements with an accuracy of ±0.4% of the current or ±3 pA, whichever is greater.

The early sampled voltages give imperfect but timely estimates of how quickly the capacitor is charging and thus of the likely current from the amperometric sensor whereas the sampled voltages from previous measurement cycles give precise but out of date estimates. From the early sampled voltage and/or the sampled voltage from previous measurement cycles, an optimised time at which the final voltage sampling in the current measurement cycle should be made to avoid reaching RIC saturation but for the capacitor to be sufficiently charged to give a strong signal to noise ratio is derived. Thus when the final sampled voltage is made at this optimum time within each cycle, more precise estimates of the current can be obtained. That is, the optimum time is a time where the RIC is not saturated and preferably some time before saturation to provide for some headspace to account for imperfections in the rough estimates based on any imperfect estimates used to set the optimised sample time. Once the final sampled voltage has been taken, the RIC can be reset, the capacitor discharged, and the measurement cycle ended. When it is time for the next measurement cycle to start, for example at the start of the next 60 second window, the cycle is repeated.

In this way, smaller and more comfortable analyte sensors that are typically considered inaccurate due to low current outputs may be used to provide highly accurate measurements in a power efficient way, thus improving comfort for users who have to implant such sensors into their skin.

Typically, measuring amperometric currents in known analyte monitoring systems is performed by utilising a transimpedance amplifier (TIA), which acts as a current to voltage converter. This approach can sample at high frequencies but has several disadvantages over the present disclosure.

At lower currents, such as those in the pico-amp to nano-amp range, the signal must be amplified to a level compatible with an analogue to digital converter's input requirements. This requires signal amplification (gain) of 109 or more with multiple amplifier stages and\or programmable gain amplifiers and digital-to-analogue converters to boost and offset the signal, each of which add to the component count, board space, error budget, and circuit power consumption.

As low-level signals are prone to having a significant noise component from radio frequency and mains interference, the signal to noise ratio (SNR) becomes significant. Thus a combination of higher sample rates, analogue filtering, digital signal processing (dsp), oversampling and averaging must be employed to reduce the SNR to acceptable levels, further increasing the component count, cost, board space requirements and power consumption. Digital processing can be particularly energy intensive, potentially needing a higher specification (and more costly) microprocessor to handle the computational burden.

These factors increase device size and power budget, with an option of larger batteries for extended operation or smaller batteries and reduced operating times and frequent battery replacement or recharging. These are undesirable in ergonomic wearable sensors, the user preference being small size and longer operating times.

In contrast, using a RIC and dynamically setting the final voltage sample timing to obtain the current estimates provides the following advantages over known systems, particularly for applications featuring low-frequency signal measurement (i.e. intermittently sampling of the order of around a minute or more as opposed to continuous monitoring):
Very high sensitivity at low currents
Low measurement noise.
Wide dynamic range (spanning multiple decades) with a single hardware and without adjusting gain or offsets or switching components in or out.
Minimal or no digital signal processing.
Low component count
Small PCB size
Low cost components.
Microprocessor can be put in a low power mode between samples
Low power consumption.
Extended run time (for example >14 days on a single, small sized battery)
No need to recharge, or replace the battery during usage.
Smaller ergonomic device size of body worn sensor enabled by small battery and small low cost circuitry components.
Less calibration complexity over comparable conventional TIA measurement systems.

Thus, according to a first aspect, there is provided a current measurement device for an analyte monitoring system, the measurement device comprising: a skin-implantable amperometric sensor connected to a RIC, the RIC comprising a capacitor configured to be charged by an electrical current flowing from the amperometric sensor; a voltage measurement system (for example an ADC) configured to sample an RIC output, the sampled voltage being indicative of the charge in the capacitor and indicative of a current flowing from the amperometric sensor into the RIC; and a reset switch configured to discharge the capacitor. The measurement device further comprises a controller configured to: (i) start a measurement cycle and begin charging the capacitor, and (ii) end the measurement cycle by resetting the RIC and discharging the capacitor.

Optionally, the controller is configured to: sample the voltage at the capacitor at a first time, during the measurement cycle while the capacitor is being charged, to make a first current estimate, the sampled voltage at the first time being indicative of a charging rate of the capacitor and of the current flowing from the amperometric sensor into the RIC between the start of the measurement cycle and the first time.

Optionally, the controller is configured to: sample the voltage at the capacitor at a second time, after the first time and during the measurement cycle while the capacitor is being charged, to make a second current estimate, the sampled voltage at the second time being indicative of a charging rate of the capacitor and of the current flowing from the amperometric sensor into the RIC between the first time and the second time.

Optionally, the controller is configured to output the second current estimate as a measurement result of the current measurement device.

Optionally, the total time of the measurement cycle is substantially 60 seconds.

Optionally, the controller is configured to set the second time based on the sampled voltage (or voltage slope) at the first time. It will be understood that the voltage slope may also be referred to as a rate of change of the voltage.

Advantageously, the sampled voltage (or voltage slope) at the first time provides a rough (i.e. inaccurate) but timely indication of how fast the capacitor is charging (e.g. a dV/dt value between the start of the measurement cycle and the first time) which is indicative of the current flowing from the amperometric sensor and this can accordingly be used to estimate when the RIC will saturate, and thus when to set the second time for. Namely, the second time can be set at when a predetermined capacitor charge level (before the RIC saturates) will be reached based on the charge rate at the first time.

Optionally, the controller is configured to set the second time based on sampled voltage (or voltage slope) from a previous measurement cycle.

Advantageously, the sampled voltage (or voltage slope) from a previous measurement cycle (for example, the sampled voltage (or voltage slope) at a second time of the previous measurement cycle) provides a highly accurate estimate of how fast the capacitor was charging in the previous cycle and, as typical signals from analyte sensors change slowly, this gives a good indication of how fast the capacitor will charge in the current measurement cycle. Accordingly, the second time can be set at when a predetermined capacitor charge level (before the RIC saturates) will be reached based on the charge rate from the previous cycle.

Optionally, the controller is configured to set the second time based on a weighted mean of the sampled voltage at the first time and the sampled voltage from a previous measurement cycle.

Advantageously, using the sampled voltage (or voltage slope) from the first time, early on in the measurement cycle, which is inaccurate but timely, and the sampled voltage (or voltage slope) from a previous cycle, which is accurate but not timely, together allows an estimate of charging rate of the capacitor to be obtained that is more accurate than using either sampled voltages (or voltage slopes) alone. This provides a more accurate estimate of when the RIC will reach saturation and thus when to set the second time to provide the optimum measurement time.

Optionally, the weights applied to the sampled voltage at the first time and the sampled voltage from a previous cycle when calculating the weighted mean are based on the magnitude of said sampled voltages (or voltage slopes), wherein the higher a magnitude, the higher a weight is applied to the sampled voltage at the first time compared to the sampled voltage from a previous measurement cycle.

Advantageously, when currents are higher, the capacitor charges more quickly and the sampled voltage (or voltage slope) at the first time is likely to be accurate enough on its own (i.e. it will likely be timely and accurate) without needing to consider the sampled voltage (or voltage slope) from a previous cycle (which is accurate but not timely). As such, the higher the sampled voltage (or voltage slope) at the first time, the more weight it is given when calculating the weighted mean.

Optionally, the controller is configured to perform a plurality of said measurement cycles whereby, over successive measurement cycles, the second time set by the controller converges towards a constant value for a given magnitude of current flowing from the sensor into the RIC.

Optionally, the constant value to which the second time converges over successive measurement cycles corresponds to a time at which the capacitor is substantially 70% charged.

Advantageously, the dynamic determining by the controller of where to set the second time effectively acts as a feedback loop that causes the second time to converge to a constant value for a given, constant current from the amperometric sensor. Thus, even when there are sudden changes or artefacts in the current signal, the second time will consistently converge to the optimum time over a sufficiently large number of successive cycles. For example, if the current changes and there are a small number of measurement cycles where the second time does not result in an optimum reading (e.g. because the capacitor inadvertently saturates from a higher current or the signal is too weak from a lower current), the second time will gradually be dynamically changed by the controller to reach the new optimum time for that new higher or lower current.

Optionally, the controller is configured to set the first time to a predetermined value for a first measurement cycle.

Optionally, the controller is configured to set the first time for successive measurement cycles based on at least one of a sampled voltage from a previous measurement cycle and a calculated rate of change of the voltage from the previous measurement cycle. For example, the controller may be configured to set the first time for successive measurement cycles based on the calculated ΔV/At from the previous measurement cycle.

Optionally, over successive measurement cycles, the first time set by the controller converges towards a constant value for a given magnitude of current flowing from the sensor into the switched capacitor circuit corresponding to a time at which the capacitor is substantially 10% charged.

Advantageously, as above with the second time, the controller also dynamically sets the first time which accordingly converges to an optimum time for a given current. Even where that current changes, and the first time is not optimum for a few cycles (e.g. because of a sudden increase or decrease in current), it will eventually converge in the same way that the second time converges as described above.

Optionally, the controller is configured to set an upper limit for the second time within the measurement cycle when the sampled voltage at the first time and/or, where applicable, the sampled voltage from a previous measurement cycle is below a threshold value.

Optionally, the upper limit for the second time corresponds to substantially 90% of the total time of the measurement cycle.

Optionally, for the total measurement cycle time of 60 seconds, the upper limit is substantially 55 seconds.

Advantageously, where a very low current would result in the second time being set to be outside of the total measurement cycle time, the controller ensures the second time remains within the measurement cycle by applying an upper limit for the second time, whenever the input current is below a threshold. In the case of a 60 second measurement cycle, the upper limit may be substantially 55 seconds, which provides around 5 seconds of headspace at the end of the cycle for the capacitor to discharge and to account for any inaccuracies in the sampled voltage.

Optionally, when the second time is based on a sampled voltage from a previous measurement cycle, the controller is configured to set the second time to correspond to a time of a 40% increase in the sampled voltage of the current measurement cycle.

Advantageously, this allows the device to accommodate sudden increases or decrease in current. For example, if there is a 40% increase or decrease in current from cycle to cycle, the second time is set to accommodate the shift of where the first and/or second time will be within the range the upper limit (and lower limit described below).

Optionally, the controller is configured to set the second time to a lower limit within the measurement cycle when the sampled voltage at the first time and/or, where applicable, the sampled voltage from a previous measurement cycle is above a threshold value.

Optionally, for the total measurement cycle time of 60 seconds, the lower limit is substantially 200 milliseconds.

Advantageously, the lower limit is similar to the upper limit in that it ensures the second time is not set too soon in the measurement cycle, for example it must not be set before the first time. This ensures the second time always remains within a predefined range.

Optionally, the controller is configured to detect that the capacitor has saturated by determining that a voltage sampled by the voltage sensor is above a threshold.

Advantageously, detecting that the capacitor has reached saturation allows the controller to provide a warning indication, for example, causing the device to indicate that any output reading is likely to be inaccurate and thus invalid.

Optionally, the controller is configured to calculate a quality metric for the first current estimate and the second current estimate by determining a difference between a sampled voltage from a previous cycle and a sampled voltage at the first time and/or the second time of the current measurement cycle, whereby the higher the difference the lower the value of the quality metric.

Advantageously, as the first time and second time converge over successive cycles to respective optimum times for given current signals, whenever the controller starts to dynamically set the first and/or second time to a times that are different to these optimum times, it is an indication that the current signal is changing and that the next few measurements are likely to be less high quality (i.e. less accurate), at least until the current has settled and the first and second times have converged to respective new values. Thus, the controller can identify from a growing difference in sampled voltages (or voltage slopes) between cycles or from differences between the first/second times of a previous cycle versus the current cycle that the quality/accuracy of the estimated current is likely to be low. For example, the greater the differences, the lower the quality metric. Where the quality metric is particularly low, it may be indicative of a hardware error or user error and may be used to notify a user of the device.

Optionally, the controller is configured to set a minimum time interval between activating the reset switch and starting a new measurement cycle to allow the capacitor to fully discharge between measurement cycles.

Advantageously, allowing sufficient time for the capacitor to discharge between cycles ensures that there is no charge left in the switched capacitor circuit that might otherwise cause inaccuracies in sampled voltages

According to a further aspect of the present disclosure, there is provided a current measurement device, the measurement device comprising: an amperometric sensor connected to a RIC, the RIC comprising a capacitor configured to be charged by an electrical current flowing from the amperometric sensor, a voltage measurement system (such as an ADC) configured to sample a voltage at the RIC output, the sampled voltage being indicative of the charge in the capacitor and indicative of a current flowing from amperometric sensor into the RIC, and a reset switch configured to reset the RIC and discharge the capacitor. The measurement system further comprises a controller configured to: (i) start a measurement cycle and begin charging the capacitor, and (ii) end the measurement cycle by resetting the RIC and discharging the capacitor

Optionally, the current device is configured for use in an environmental monitoring system.

It will be appreciated that the advantages described above in connection with the current measurement device for an analyte monitoring system apply to the corresponding features of the general current measurement device described above.

Additionally, it is envisaged that the general current measurement device may be used for environmental monitoring systems, for example to assist in estimating the concentration of pollutants or other particles in an environment, whereby the concentration of a pollutant or other particles in an environment may be estimates from a current generated by an amperometric sensor configured to detect such pollutants or particles.

### BRIEF DESCRIPTION OF THE DRAWINGS

These are and other aspects will now be described in relation to the Figures in which:

FIG. 1 illustratively shows a sensor module of an analyte monitoring system according to the present disclosure.

FIG. 2 illustratively shows a current measurement device according to the present disclosure.

FIG. 3a shows a plot of a current measurement estimation made using a device according to the present disclosure.

FIG. 3b shows a plot of a current measurement estimation made using a device according to the present disclosure.

FIG. 4 shows an illustrative plot of a capacitor charge cycle of a RIC showing voltage sample reading against time as the capacitor is charged.

FIG. 5a illustratively shows a circuit diagram implementing part of a current measurement device according to the present disclosure.

FIG. 5b illustratively shows a circuit diagram implementing part of a current measurement device according to the present disclosure.

FIGS. 6a and 6b illustratively show plots of battery life of a current measurement device according to the present disclosure.

FIG. 7 illustratively shows a graph of example methods usable by a current measurement device according to the present disclosure.

FIG. 8 illustratively shows a graph of example methods usable by a current measurement device according to the present disclosure at different sensor current ranges.

FIG. 9 depicts example current measurements from a current measurement device according to the present disclosure.

### DETAILED DESCRIPTION

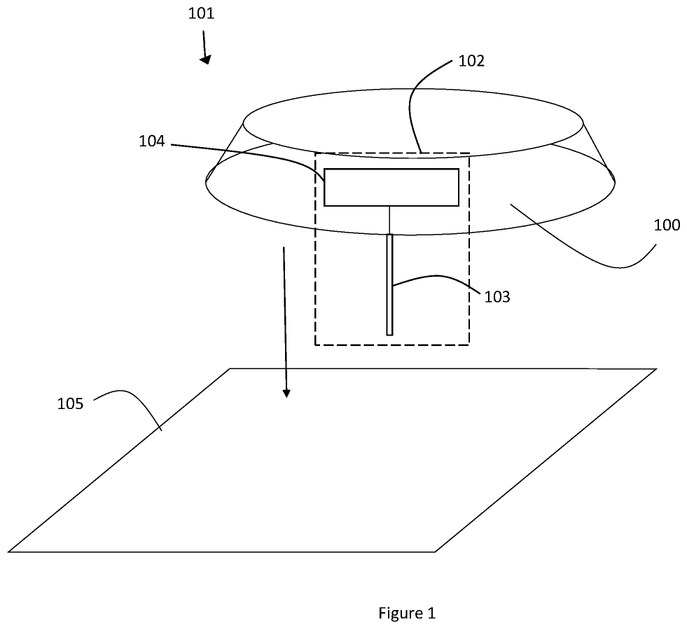

FIG. 1 illustratively shows a sensor module 100 of an analyte monitoring system 101 according to the present disclosure. The sensor module 100 comprises current measurement device 102 comprising a skin-implantable amperometric sensor 103 and measurement circuitry 104 comprising a RIC and a controller, for example a microprocessor. In use, the sensor module 100 is placed on the surface of the skin 105 of the user and the skin-implantable amperometric sensor 103 is implanted into the user's skin 105 where it will generate an output current signal based on the analyte being measured.

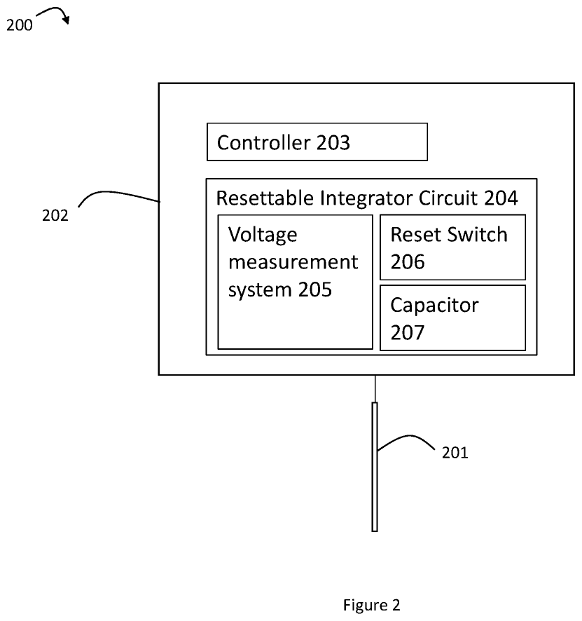

FIG. 2 illustratively shows a current measurement device 200 according to the present disclosure, such as current measurement device 102 of FIG. 1. The current measurement device 200 comprises a skin-implantable amperometric sensor 201 and measurement circuitry 202. The measurement circuitry 202 comprises a controller 203 and a RIC 204 and a voltage measurement system 205. The RIC 204 comprises a voltage measurement system 205, a capacitor 207 and a reset switch 206. The reset switch m206 may also be referred to as a discharge switch. The capacitor 207 is configured to be charged by an electrical current flowing from the skin-implantable amperometric sensor 201 and configured to be discharged upon activation of the reset switch 206 to thereby reset the charge cycle of the capacitor 207. The voltage sensor 205 is configured to sample a voltage of the capacitor 207, the sampled voltage being indicative of a charging rate of the capacitor 207 over a first time period and indicative of a current flowing from the skin-implantable amperometric sensor 201 during said first time period.

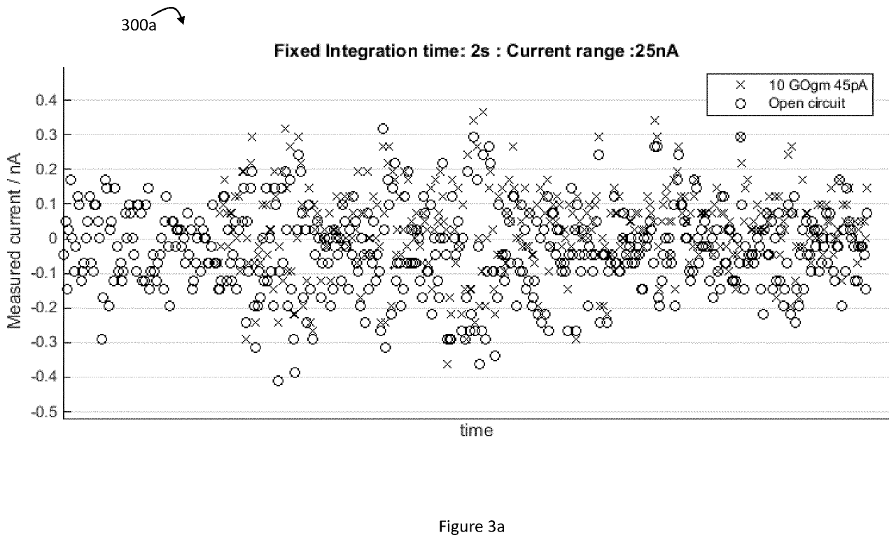

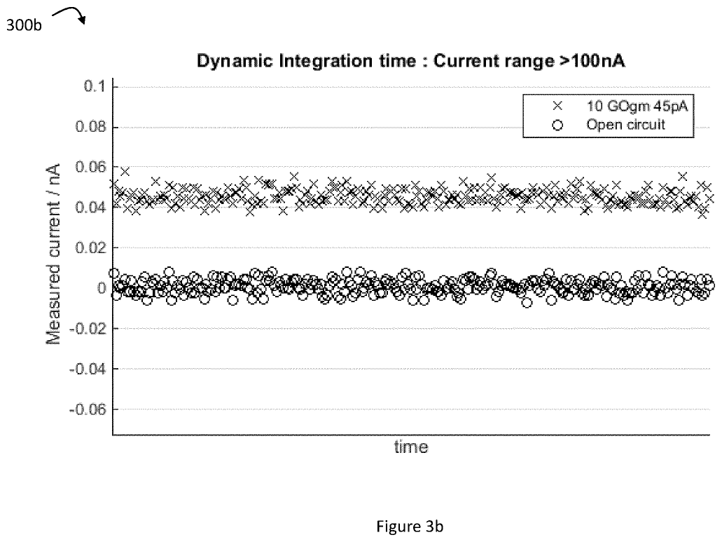

FIGS. 3a and 3b respectively show plots 300a, 300b of a current measurement estimation made using a device according to the present disclosure, whereby a low, continuous output of an amperometric sensor is simulated by using a using a 10 gigaOhm resistor to produce a 45 pA current, data is also collected from a fully open circuit (i.e. 0 current) to provide a baseline to compare to. The device of the present disclosure effectively operates like integrator circuit and each data point represents 60 second measurement cycle. The RIC saturation level used to produce the plots was set at around 100 nA.

In FIG. 3a, integration time (i.e. when the voltage is sampled, the current estimated and the switched capacitor circuit reset) is not set dynamically but is fixed at 2 seconds. It is apparent from FIG. 3a that it is difficult to distinguish between the 45 pA current signal and the open circuit base line in this setup.

In contrast, in FIG. 3b, integration time (i.e. when the voltage is sampled, the current estimated and the switched capacitor circuit reset) is set dynamically and it is immediately apparent from FIG. 3b that the noise of FIG. 3a has effectively disappeared and it is substantially easier to distinguish between the baseline open circuit values and the low 45 pA simulated test current signal. As a result, a current measurement device of the present disclosure will be able to far more accurately measure current signals in the pico amp range compared to known devices of analyte monitoring systems, and it is able to do so in a power efficient way without any separate, power hungry digital signal processing.

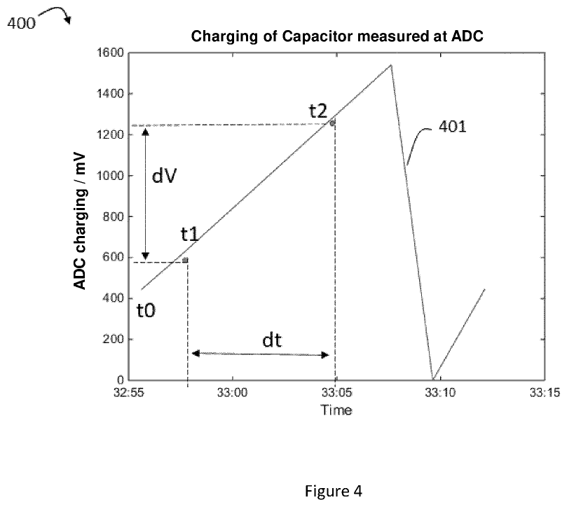

FIG. 4 illustratively shows a plot 400 of a capacitor charge cycle 401 of a capacitor of a RIC showing expected voltage sample reading against time as the capacitor is charged by a current signal and then reset. FIG. 4 is provided to illustrate how measuring a preliminary voltage (i.e. a voltage early on in the capacitor charge cycle) can be used to estimate when RIC saturation level is likely to be reached and thus when to optimally time the voltage measurement indicative of the current from the amperometric sensor. Specifically, for a given measurement cycle with at t0, the RIC voltage output may be measured shortly after the start at t1. This provides a slope of the line dV/dt indicating a rough estimate of the charge rate of the capacitor. As the capacitance of the capacitor and RIC properties are known from the design of the device, this gives an indication of how long it will take before RIC saturation is reached and how long the window is for the controller of the device to cause the voltage measurement system to sample the voltage reset the circuit. In the example of FIG. 4, some headspace is given so that the latest time is some distance away from saturation levels, for example at t2. This helps to compensate for any inaccuracies in the measurement of the voltage at t1.

As will be appreciated from FIG. 4, dynamically adjusting t2 (i.e. the time when the voltage is sampled and the final result of that measurement cycle is estimated) ensures that the reading is always at an optimum time during the measurement cycle. If it is too early the reading will be noisy and imprecise because the capacitor has not had sufficient time to charge to produce a strong signal to noise ratio in the voltage reading. Conversely, if it is too late in a measurement cycle it is likely to be around when the RIC is already saturated which also leads to an inaccurate reading. Thus, the device of the present disclosure is particularly effective at producing an accurate reading in the context of analyte monitoring systems in human bodies where the current output by an implanted amperometric sensor will be very low, inconsistent and prone to noise.

Whilst not shown in FIG. 4, a corresponding technique may be used to identify when to set t2 of a current measurement cycle but now using the sampled voltage from a t2 of a previous measurement cycle (or one or more parameters derived therefrom, for example the estimated current and/or the slope dV/dt of the previous cycle. These values from one or more previous charge cycles will typically be similar to a current cycle because the input current signals from analyte monitoring systems often do not change quickly. For example, if the slope dV/dt is of a previous charge cycle is first value, then this value is taken as an estimate of the slope dV/dt of the current cycle and may be used accordingly to estimate where the RIC will saturate and accordingly when the optimum time for t2 will be to sample the voltage at the capacitor to estimate the current from the sensor in this cycle.

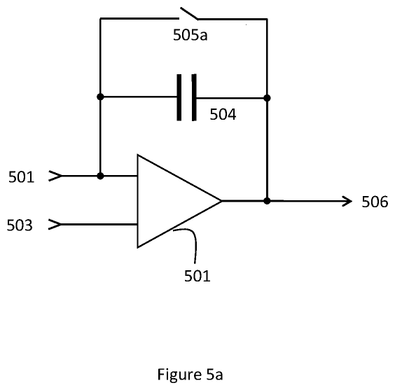

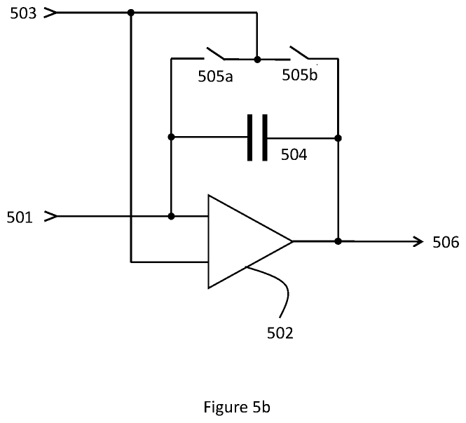

FIGS. 5a and 5b illustratively show simplified circuit diagrams of parts of respective current measurement devices according to the present disclosure.

The circuit of FIG. 5a shows an example RIC configuration. It comprises a current input line 501 connected to the inverting input of an operational amplifier 502, an electrode potential line 503 connected to the non-inverting input of the operational amplifier 502, a capacitor 504 connected at one side to the current input line 501 and on the other to the output of the operational amplifier 506. A single reset switch 505a is also provided and connected in parallel with capacitor 504. Output line 506 may be fed into an analogue to digital converter (ADC) or other voltage measurement system for further processing of the signal. The circuit operates as described above. That is, while the switch 505a is open, the current from the amperometric sensor of e.g. FIGS. 1 and 2 charges the capacitor which results in a potential difference across the capacitor 504 which is proportional to the amount of charge stored and thus proportional to the magnitude of the current flowing form the amperometric sensor and thus indicative of a concentration of an analyte. The change in potential difference across the capacitor 504 is detectable in the output 506. Activating the reset switch 505a discharges capacitor 504 and occurs at the end of a measurement cycle.

FIG. 5b corresponds to that of FIG. 5a except that a second reset switch 505b is also provided in series with the first reset or discharge switch 505a and the electrode potential line 503 is not only connected to the non-inverting input of the operational amplifier 502 but also the junction of two reset switches, 505a and 505b. During operation, both switches 505a, 505b operate as one (that is, to reset the circuit and discharge the capacitor 504, they activate together). It will be understood that additional reset switches may also be provided, for example such that the RIC comprises three, four, five or more reset switches.

The circuit in FIG. 5b may be less susceptible to current leakage and switching artefacts than that of FIG. 5a, while the circuit of FIG. 5a is simpler than the circuit of FIG. 5b and so is more easily manufactured. In certain implementations, the use of two serially-connected switches (505a and 505b) in series in FIG. 5b, with the mid-point connected to the electrode potential: (i) equalises the potential applied to across the switches when open, minimising the effective leakage current; and (ii) equalises and maintains the potential across the switches and input 501 when closed, reducing switching artefacts appearing at output 506.

It is envisaged that the current input line 501 in both FIGS. 5a and 5b is connected to an output of the amperometric sensor described above. Additionally, FIGS. 5a and 5b do not show any resistive loads but it is envisaged that these will be present as will be appreciated by the skilled person. The switches of FIGS. 5a and 5b may be discrete devices or integrated into an analogue switch chip on a single, solid state, substrate, and controlled by the controller of the current measurement device described above.

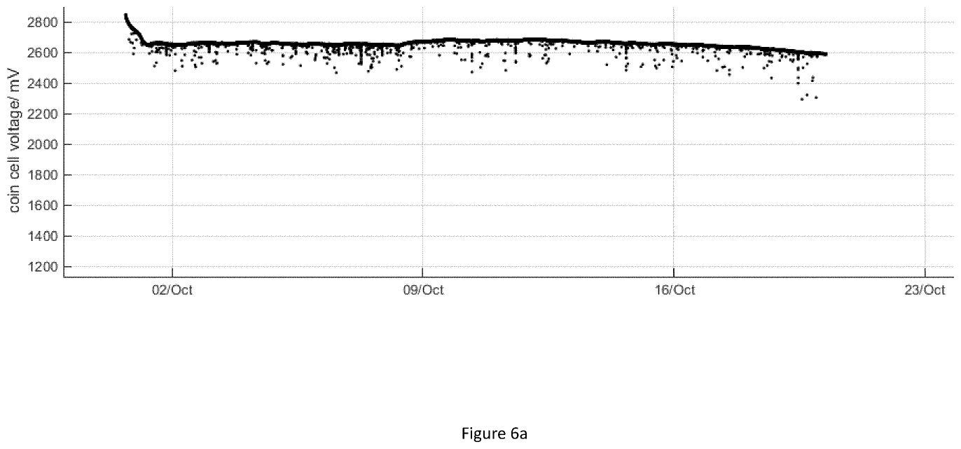

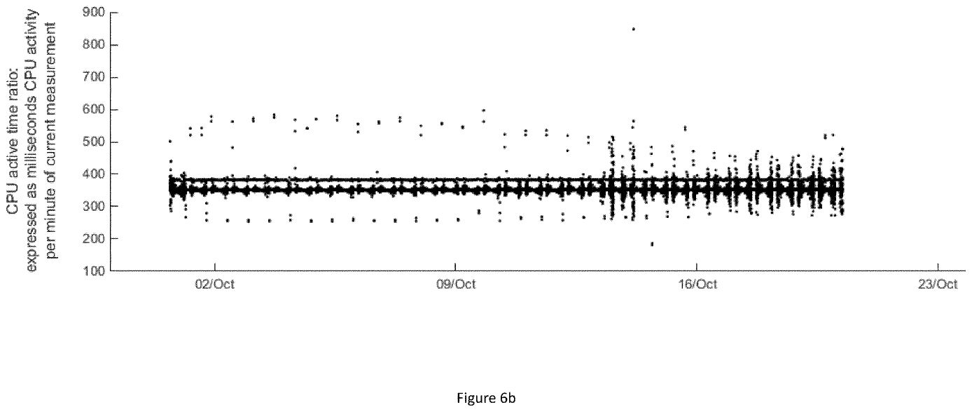

FIGS. 6a and 6b show plots respectively demonstrating how battery life of a current measurement device according to the present disclosure extends to at least 20 days as a result of the device having a reduced processing overhead requirement. Specifically, the on-time of the processing resources used in the present device is substantially less than in known devices because higher sample rates, analogue filtering, digital signal processing (dsp), oversampling and averaging to increase signal to noise ratio are not required as a result of the dynamic setting of the measurement cycle timing as described herein.

Specifically, FIG. 6a shows a plot of battery output voltage of a coin cell battery in mV over time during continuous operation of a current measurement device according to the present disclosure. The device is switched on at an example date of around 1 October and it can be seen that the battery continues to output around 2600 mV around 20 days later on 20 October indicating that the current measurement device can operate for an order of magnitude of tens of days up without the battery running out. In contrast, known devices typically require a recharge or replacement battery after around 7 hours or an order of magnitude of hours rather than tens of days. The occasional minor drops in battery output may occur typically at times when the processor is sampling the current and/or activating the reset or discharge switch(es), and it is apparent that these are relatively spread out over the time period of around 20 days. For example, the measured battery output voltage may drop when sampled or when computed results are transferred to external devices, for example via Bluetooth® or any other suitable wired or wireless communication standard. Voltage drops resulting from the use of communications such as Bluetooth® may be particularly noticeable when the battery of the device is largely depleted. It will be understood that the system circuitry may be configured to accommodate this behaviour and to avoid or otherwise minimise voltage drops in the supply to the RIC, to thereby reduce the impact of the voltage drops on measurement data. For example, an RIC supply may be below the battery end-of-life potential (e.g. below 2.00V) and stable. In one example, the RIC supply may be 150 mV.

A primary reason for this extended battery lifetime compared to known devices is that the microprocessor “on-time” is very low due to the dynamic setting of timing of the measurement cycle which increases the total time the microprocessor is able to spend in a low-power, sleep state, between measurement cycles. Specifically, FIG. 6b shows a plot of the same time period as FIG. 6a but now showing a CPU active time ratio expressed as milliseconds of CPU activity per minute of current measurement. The CPU is in a low-power, sleep state, idling at a CPU active time ratio of between 300-400. The occasional spikes to a CPU active time ratio of around 500-600 typically occur when the current is being sampled/measured and the reset or discharge switches activated. Again, it can be seen from FIG. 6b that this occurs generally rarely during the 20 day time period and this accordingly facilitates the battery lasting substantially longer (for example at least 20 days as shown in FIG. 6a) than compared to known devices.

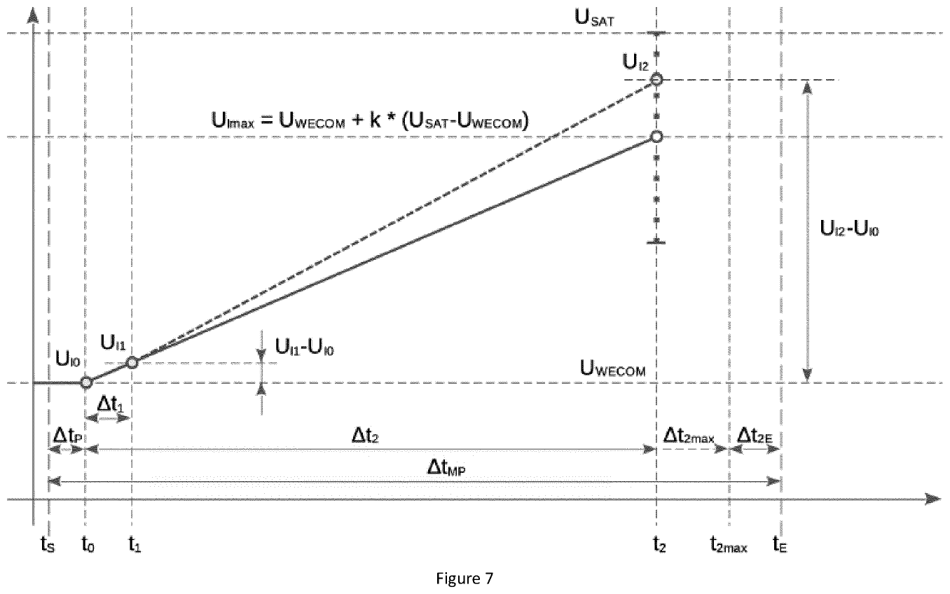

FIG. 7 illustratively shows a graph illustrating a detailed implementation of the methods used by a current measurement device according to the present implementation. The example of FIG. 6 is provided for illustrative purposes only and it will be appreciated that other ways of implementing the present disclosure are also envisaged.

The graph of FIG. 7 is similar to the simplified graph shown in FIG. 4 showing a measurement cycle wherein a time period is shown during which the capacitor of the present disclosure starts charging at t0 and is eventually discharged at the end of the measurement cycle at t2.

Specifically, as is shown in FIG. 7, the sensor current measurement is contained within the Measurement Period Δ_{tMP}.

The sensor current measurement starts with an intermission time (pause) Δt_{P}, after the start of the measurement period t_{S}, to ensure any leftover parasitics from the previous cycle have ended thus guaranteeing the discharge of the integrating capacitor from the previous measurement cycle.

The integration starts after this intermission time and immediately a point data (PD) sample is taken. Its value is denoted by U_{I0} and its timestamp t₀.

At time t1=t0+Δt another PD is sampled. Its value is denoted by U₁₁. The time Δt₁ depends on the previously measured slope of the integration line Sₚᵣₑᵥ. If no such value is available, Δt₁ is set to a default value. Otherwise, it is computed so that an expected increase (a parameter) ΔU_{I1} of the voltage at the integrator output for this time is achieved:

$$\\ Δ {t}_{1} = \frac{Δ {U}_{I 1}}{{S}_{prev}}$$

This expected increase parameter may also be limited to a predefined range [Δt₁ₘᵢₙ, Δt₁ₘₐₓ].

Thus, an initial slope of the integration line is computed as:

$$\\ {S}_{0} = \frac{{U}_{I 1} - {U}_{I 0}}{Δ {t}_{1}}$$

Next, the time [symbol] after which the maximum allowed voltage U_{Imax} at the integrator output would be reached, is estimated:

$$\\ {\hat{Δ t}}_{2} = \frac{{U}_{Imax} - {U}_{I 0}}{{S}_{0}}$$

The time [symbol] is used to determine the actual time Δt₂ after which (with respect to t₀), a third PD shall be sampled. The time Δt₂ is limited within the range [Δt₂ₘᵢₙ, Δt₂ₘₐₓ] where Δt₂ₘᵢₙ, =2 Δt₁ₘᵢₙ and Δt₂ₘₐₓ, =Δt_{MP}−Δt_{2E}−Δt_{P}.

If [symbol] ∈[Δt₂ₘᵢₙ,Δ₂ₘₐₓ] then Δt₂= [symbol] .

If [symbol] >Δt₂ₘₐₓ then Δt₂ is set to Δt₂ₘₐₓ. In these two cases, a third PD sampling is scheduled at time t₂=t₀+Δt₂. Its value is denoted by U_{I2}.

If [symbol] <Δt₂ₘᵢₙ then no third PD is sampled at all.

The final slope of the integration line is computed as:

$$\\ S = \frac{{U}_{I 2} - {U}_{I 0}}{Δ {t}_{2}}$$

if U_{I2} exists and as S=S₀ if U_{I2} does not exist.

The sensor current is finally obtained as a product of the slope of the integration line and the value of the integrating capacitor:

Iₛ=C_{I}S

An illustrative set of operational parameters for FIG. 7 will now be described which the inventors have found provide the advantages described above. However, it will be understood that these are illustrative only and different values may also be used to obtain corresponding advantages depending on the given needs of a system.

Intermission time Δt_{P} is a session parameter with an initial default value of 500 ms.

A maximum allowable voltage U_{SAT} at the RIC output may depend on the maximum allowable parameters of the specific component parts used for the RIC. It will be appreciated that these will be set by a manufacturer of the components and are set lower than the integrator's power supply voltage U_{AN}, a nominal value of which may be 1850 mV. It is envisaged that, to account for potential deviations, U_{SAT} may be set to 1800 mV.

A maximum allowed voltage U_{Imax} at the integrator output is derived as a percentage k of the span between U_{WECOM} and U_{SAT}, above U_{WECOM}. A default value for k may be 70% and may be based on a preliminary observed distribution of measured constant sensor currents for a given set of components used.

The default value for the time Δt₁ is a session parameter which may have an empirical default value of 1 s.

The value of Δt₁ₘᵢₙ is a session parameter with a default value of 200 ms but may also depend on and be tailored to the computational speed of a communication interface used by transmitter system to send measurement readings from the device.

The value for A U_{I1}, which defines the preferable increase of the voltage at the integrator, needed for a successful initial estimation of the integration line slope, is a session parameter, and may have a default value of 100 mV.

The value of Δₜ₁ₘₐₓ is derived from A U_{I1} and for the default value of ΔU_{I1}, the resulting value for Δₜ₁ₘₐₓ may be 4037 ms. Further details are provided below.

The time Δt_{2E} is a session parameter which may have an empirical default value of 5 s. This is the time before of the end of the measurement period in which any activity should be suspended.

The measurement period Δ_{tMP} comes from the system requirements but may be defined as a session parameter with a default value of 60 s.

The base voltage at the RIC output depends on the manufacturer specified operational parameters of the components used, for example, U_{WECOM} may have an expected value of 450 mV.

The value U_{I0} of the first PD sample is expected to be equal to the value of U_{WECOM} and can be used as a criterion for a hardware failure.

The value U_{I1} of the second PD sample is expected to be less than the U_{SAT} and can be used as a criterion for a very large sensor current (e.g. during warm-up, or if a short circuit is present).

The value U_{I2} of the third PD sample is also expected to be less than the U_{SAT} and can be used as a criterion for a large amount of noise in the sensor current (e.g. due to skin motion).

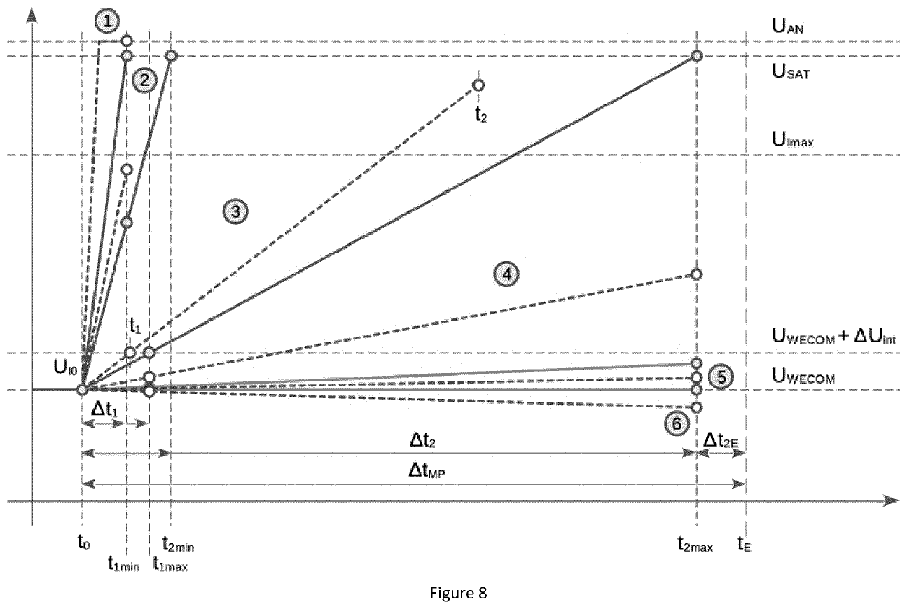

FIG. 8 illustratively shows a graph corresponding to FIG. 7 but now showing a plurality of plots corresponding to different envisaged illustrative sensor current ranges based on the above operational parameters. In FIG. 8, there are six specific current ranges but it is envisaged that any other number may be used.

The example operational current ranges illustrated in FIG. 8 are explained as follows.

Saturated high (1). The maximum sensor current, which can be evaluated is the one that does not bring saturation at time t₁ₘᵢₙ:

$$\\ {I}_{Smax 1} = {C}_{I} \frac{{U}_{SAT} - {U}_{I 0}}{Δ {t}_{1 min}} = 47 nF \cdot \frac{( 1800 mV - 450 mV )}{0.2 s} = 47 nF \times 6.75 V / s = 317.25 nA$$

Any larger current would bring saturation at the output of the RIC.

The range is defined as Iₛ∈└I_{Smax1} . . . ∞). For the default parameter settings described above, this corresponds to [317.25 nA . . . ∞).

High current (2). Sensor current, computed at time t₁ is lower than t_{Smax1} but high enough to bring saturation at t₂ₘᵢₙ where:

$$\\ {I}_{Smax 2} = {C}_{I} \frac{{U}_{SAT} - {U}_{I 0}}{Δ {t}_{2 min}} = 47 nF \cdot \frac{( 1800 mV - 450 mV )}{0.4 s} = 47 nF \times 3.375 V / s = 158.625 nA$$

In this case, a sensor current value is available, computer at the moment t1.

The range is defined as I_{S}∈[I_{Smax2} . . . I_{Smax1}). For the default parameter settings described above, this corresponds to [158.625 nA . . . 317.25 nA).

Normal-to-high current (3). This includes sensor currents lower than I_{Smax2} but larger than I̅_̅{̅S̅b̅e̅s̅t̅}̅ the value at which t2 will be scheduled to be t₂ₘₐₓ.

$$\\ \overline{{I}_{Sbest}} = {C}_{I} \frac{{U}_{Imax} - {U}_{I 0}}{Δ {t}_{2 max}} = {C}_{I} \frac{{U}_{WECOM} + k ( {U}_{SAT} - {U}_{WECOM} ) - {U}_{I 0}}{Δ {t}_{MP} - Δ {t}_{2 E} - Δ {t}_{P}} = {C}_{I} \frac{k ( {U}_{SAT} - {U}_{WECOM} )}{Δ {t}_{MP} - Δ {t}_{2 E} - Δ {t}_{P}}$$

$$\\ \overline{{I}_{Sbest}} = 47 nF \cdot \frac{0.7 ( 1800 mV - 450 mV )}{54.5 s} = 47 nF \times 0.01734 V / s = 0.815 nA$$

Thus the normal-to-high range is defined as I_{S}∈I_{Sbest} . . . I_{Smax2}).

The maximum expected random fluctuation of the current at t₂ would be:

$$\\ {I}_{Sbest} = {C}_{I} {S}_{best} = {C}_{I} \frac{{U}_{Imax} - {U}_{I 0}}{Δ {t}_{2 max}} = {C}_{I} \frac{{U}_{WECOM} + ( {U}_{SAT} - {U}_{WECOM} ) - {U}_{I 0}}{Δ {t}_{MP} - Δ {t}_{2 E} - Δ {t}_{P}} {C}_{I} \frac{( {U}_{SAT} - {U}_{WECOM} )}{Δ {t}_{MP} - Δ {t}_{2 E} - Δ {t}_{P}}$$

$$\\ {I}_{Sbest} = 47 nF \cdot \frac{( 1800 mV - 450 mV )}{54.5 s} = 47 nF \times 0.02477 V / s = 1.1642 nA$$

Normal-to-low current (4). For sensor currents lower than I̅_̅{̅S̅b̅e̅s̅t̅}̅, t₂ will be always scheduled to be equal to t₂ₘₐₓ. This range is bounded from its lower side to a value I_{Smin} that is not likely to be due to a connected sensor, i.e. I_{S}∈I_{Smin} . . . I_{Sbest}) or for the above described default parameters, I_{S}∈[0.2 nA . . . 1.164 nA).

Low current (5). I_{S}∈[0 . . . I_{Smin}) which, for the above described default parameters corresponds to I_{S}∈[0 nA . . . 0.2 nA).

Saturated low (6). I_{S}<0

It is envisaged that, during operation of the current measurement device, it will be determined which current range a given current belongs to and this may be annotated suitably with a property flag. At any time instance, if a saturation at the output of the RIC is detected or predicted, the measurement cycle is immediately stopped.

It will be appreciated that the value of Δₜ₁ₘₐₓ may be chosen so that at the slope S_{best} for I_{Sbest}, the value of U_{I1} is larger than U_{I0} with ΔU_{I1}, i.e.

$$\\ Δ {t}_{1 max} = \frac{Δ {U}_{I 1}}{{S}_{best}}$$

or, in numbers, using the above default operational parameters:

$$\\ Δ {t}_{1 max} = \frac{0.1 V}{0.02477 V / s} = 4.037 s$$

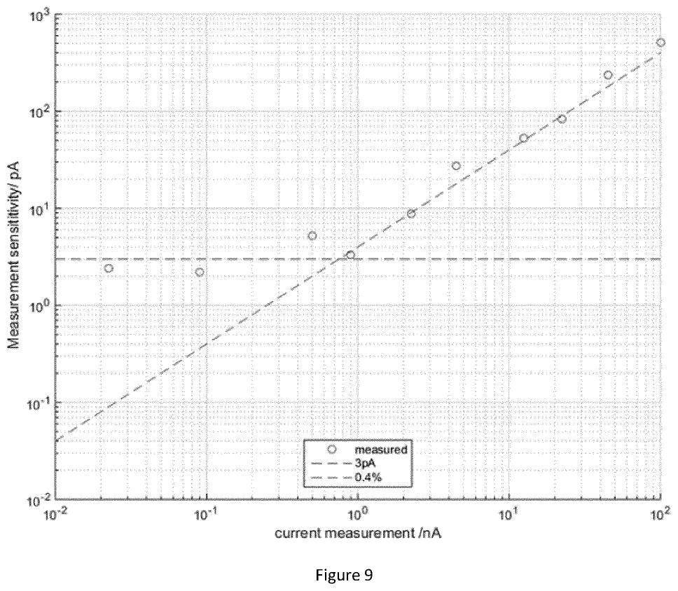

FIG. 9 illustratively depicts example current measurements from an example implementation in which the accuracy/limit of detection (p<0.05) is the greater of 3 pA or 0.4% in a range of 0 to 300 nA.

Other effective alternatives will occur to the skilled person. It will be understood that the invention is not limited to the described embodiments and encompasses modifications apparent to those skilled in the art lying within the scope of the claims appended hereto.

For example, whilst the present disclosure primarily focusses on analyte monitoring systems for use in a patient, for example to measure blood glucose or other analytes, it is envisaged that the device may also be used in any application where very small currents need to be measured at infrequent intervals, such as at minute or longer intervals. A particular envisaged use case is environmental monitoring where an amperometric sensor is used to measure pollutants or other particles and the output signal of the sensor is very low, in the pico to nanoamp range.

1: A current measurement device for an analyte monitoring system, the measurement device comprising:
a skin-implantable amperometric sensor connected to a resettable integrator circuit (RIC), the RIC comprising:
a capacitor configured to be charged by an electrical current flowing from the amperometric sensor;
a voltage measurement system configured to sample a voltage at an RIC output, the sampled voltage being indicative of the charge in the capacitor and indicative of a current flowing from the amperometric sensor into the RIC; and
a reset switch configured to discharge the capacitor; and
a controller configured to: (i) start a measurement cycle by charging the capacitor, and (ii) end the measurement cycle by resetting the RIC to discharge the capacitor.

2: The current measurement device of claim 1, wherein the controller is configured to:
sample the voltage at the RIC output at a first time, during the measurement cycle while the capacitor is being charged, to make a first current estimate, the sampled voltage at the first time being indicative of a charging rate of the capacitor and of the current flowing from the amperometric sensor into the RIC between the start of the measurement cycle and the first time.

3: The current measurement device of claim 2, wherein the controller is configured to:
sample the voltage at the RIC output at a second time, after the first time and during the measurement cycle while the capacitor is being charged, to make a second current estimate, the sampled voltage at the second time being indicative of a charging rate of the capacitor and of the current flowing from the amperometric sensor into the RIC between the first time and the second time.

4: The current measurement device of claim 3, wherein the controller is configured to output the second current estimate as a measurement result of the current measurement device.

5: The current measurement device of claim 3, wherein the total time of the measurement cycle is substantially 60 seconds.

6: The current measurement device of claim 3, wherein the controller is configured to set the second time based on the sampled voltage at the first time.

7: The current measurement device of claim 3, wherein the controller is configured to set the second time based on sampled voltage from a previous measurement cycle.

8: The current measurement device of claim 6, wherein the controller is configured to set the second time based on a weighted mean of the sampled voltage at the first time and the sampled voltage from a previous measurement cycle; and optionally wherein the weights applied to the sampled voltage at the first time and the sampled voltage from a previous cycle when calculating the weighted mean are based on the magnitude of said sampled voltages, wherein the higher a magnitude, the higher a weight is applied to the sampled voltage at the first time compared to the sampled voltage from a previous measurement cycle.

9. (canceled)

10: The current measurement device of claim 3, wherein the controller is configured to perform a plurality of said measurement cycles whereby, over successive measurement cycles, the second time set by the controller converges towards a constant value for a given magnitude of current flowing from the sensor into the RIC; and optionally wherein the constant value to which the second time converges over successive measurement cycles corresponds to a time at which the capacitor is substantially 70% charged.

11. (canceled)

12: The current measurement device of claim 3, wherein the controller is configured to set the first time to a predetermined value for a first measurement cycle.

13: The current measurement device of claim 12 wherein the controller is configured to set the first time for successive measurement cycles based on at least one of a sampled voltage from a previous measurement cycle and a calculated rate of change of the sampled voltage from the previous measurement cycle; and optionally wherein, over successive measurement cycles, the first time set by the controller converges towards a constant value for a given magnitude of current flowing from the sensor into the RIC corresponding to a time at which the capacitor is substantially 10% charged.

14. (canceled)

15: The current measurement device of claim 3, wherein the controller is configured to set an upper limit for the second time within the measurement cycle when the sampled voltage at the first time and/or, where applicable, the sampled voltage from a previous measurement cycle is below a threshold value; and optionally wherein the upper limit for the second time corresponds to substantially 90% of the total time of the measurement cycle.

16. (canceled)

17: The current measurement device of claim 15, for the total measurement cycle time of 60 seconds, the upper limit is substantially 55 seconds.

18: The current measurement device of claim 3, wherein, when the second time is based on a sampled voltage from a previous measurement cycle, the controller is configured to set the second time to correspond to a time of a 40% increase in the sampled voltage of the current measurement cycle.

19: The current measurement device of claim 3, wherein the controller is configured to set the second time to a lower limit within the measurement cycle when the sampled voltage at the first time and/or, where applicable, the sampled voltage from a previous measurement cycle is above a threshold value; and optionally wherein for the total measurement cycle time of 60 seconds, the lower limit is substantially 200 milliseconds.

20. (canceled)

21: The current measurement device of claim 1, wherein the controller is configured to detect that the RIC has saturated by determining that a voltage sampled by the voltage sensor is above a threshold.

22: The current measurement device of claim 1, wherein the controller is configured to calculate a quality metric for the first current estimate and the second current estimate by determining a difference between a sampled voltage from a previous cycle and a sampled voltage at the first time and/or the second time of the current measurement cycle, whereby the higher the difference the lower the value of the quality metric.

23: The current measurement device of claim 1, wherein the controller is configured to set a minimum time interval between activating the reset switch and starting a new measurement cycle to allow the capacitor to fully discharge between measurement cycles.

24: A current measurement device, the measurement device comprising:
an amperometric sensor connected to a resettable integrator circuit (RIC), the RIC comprising:
a capacitor configured to be charged by an electrical current flowing from the amperometric sensor;
a voltage measurement system configured to sample a voltage at an RIC output, the sampled voltage being indicative of the charge in the capacitor and indicative of a current flowing from amperometric sensor into the RIC; and
a reset switch configured to reset the RIC and discharge the capacitor; and
a controller configured to: (i) start a measurement cycle by charging the capacitor, and (ii) end the measurement cycle by resetting the RIC and discharging the capacitor.

25: The current measurement device of claim 24, configured for use in an environmental monitoring system.
